(19) United States
(12) Patent Application Publication
(10) Pub. No.: US 2014/0271604 A1
(43) Pub. Date: Sep. 18, 2014

(54) HDAC Inhibitor Polymorphic Forms and Methods of Use

(71) Applicant: STEFAN PRONIUK, San Diego, CA (US)

(72) Inventor: STEFAN PRONIUK, San Diego, CA (US)

(21) Appl. No.: 14/203,808

(22) Filed: Mar. 11, 2014

Related U.S. Application Data
(60) Provisional application No. 61/777,734, filed on Mar. 12, 2013.

(51) Int. Cl. C07C 259/10; A61K 45/06; A61K 31/185

(52) U.S. Cl. CPC C07C 259/10; A61K 31/185; A61K 45/06
USPC 424/94.6; 562/622; 514/575; 514/171; 514/400

(57) ABSTRACT

Polymorphic forms of histone deacetylase inhibitors (HDAC) and methods of making and using such polymorphic forms are provided. Crystalline polymorphic forms can be characterized by their X-ray powder diffraction patterns, solubility, stability and other properties.

### PRIORITY CLAIM

This application claims priority to U.S. Provisional Patent Application Ser. No. 61/777,734, filed Mar. 12, 2013. The above referenced application is incorporated herein by reference as if restated in full.

### BACKGROUND

Acetylation of core histones plays an important role in the regulation of gene transcription by controlling nucleosomal packaging of DNA. Deacetylation of histones results in tight packing of nucleosomes and transcriptional repression due to limited access of transcription factors to DNA targets. Histone acetylation relaxes nucleosome structures, providing greater access for transcription factors. The balance between histone deacetylation and acetylation is modulated by the histone deacetyl-transferases (HDACs) and histone acetyl-transferases (HAT). An abnormal balance of these factors is correlated with abnormal cell growth and several forms of cancer as discussed in U.S. Pat. No. 8,318,808, incorporated by reference herein in its entirety. HDAC inhibitors, in particular, change the balance between acetylation and deacetylation resulting in growth arrest, differentiation, and apoptosis in many tumor cell types. U.S. Pat. No. 8,318,808.

Of particular interest herein are HDAC inhibitors described in U.S. Pat. No. 8,318,808 and are based on, for example, fatty acids coupled with Zn²⁺-chelating motifs through aromatic Ω-amino acid linkers. In various aspects, the HDAC inhibitors may have the formula:

[chemical structure]

wherein X is chosen from H and CH3; Y is (CH2)n wherein n is 0-2; Z is chosen from (CH2)m wherein m is 0-3 and (CH)2; A is a hydrocarbyl group; B is o-aminophenyl or hydroxyl group; and Q is a halogen, hydrogen, or methyl. One HDAC inhibitor of particular (N-hydroxy-4-(3-methyl-2-phenyl-butyrylamino)-benzamide) is also known as AR-42. In one aspect, the structure of AR-42 is as follows:

[chemical structure]

AR-42 is a broad-spectrum deacetylase inhibitor of both histone and non-histone proteins with demonstrated greater potency and activity in solid tumors and hematological malignancies when compared to vorinostat (i.e., SAHA). See, e.g., Lu Y S, et al., Efficacy of a novel histone deacetylase inhibitor in murine models of hepatocellular carcinoma, Hepatology. 2007 October; 46(4):1119-30; Kulp S K, et al., Antitumor effects of a novel phenylbutyrate-based histone deacetylase inhibitor, (S)-HDAC-42, in prostate cancer, Clin Cancer Res. 2006 Sep. 1; 12(17):5199-206.

AR-42 may also possess additional histone-independent mechanisms which contribute to its therapeutic profile. See, e.g., Chen M C, et al., Novel mechanism by which histone deacetylase inhibitors facilitate topoisomerase IIa degradation in hepatocellular carcinoma cells, Hepatology. 2011 January; 53(1):148-59; Chen C S, et al., Histone acetylation-independent effect of histone deacetylase inhibitors on Akt through the reshuffling of protein phosphatase 1 complexes, J Biol. Chem. 2005 Nov. 18; 280(46):38879-87; Yoo C B, et al., Epigenetic therapy of cancer: past, present and future, Nat Rev Drug Discov. 2006 January; 5(1):37-50.

AR-42 has a demonstrated inhibitory effect in tumors including, but not limited to, breast, prostate, ovarian, blood cell (e.g., lymphoma, myeloma, leukemia), liver, and brain. See, e.g., Mims A, et. al., Increased anti-leukemic activity of decitabine via AR-42-induced upregulation of miR-29b: a novel epigenetic-targeting approach in acute myeloid leukemia, Leukemia. 2012 Nov. 26. doi: 10.1038/leu.2012.342. [Epub ahead of print]; Burns S S, et al., Histone deacetylase inhibitor AR-42 differentially affects cell-cycle transit in meningeal and meningioma cells, potently inhibiting NF2-deficient meningioma growth, Cancer Res. 2013 Jan. 15; 73(2):792-803; Lu Y S, et. al., Radiosensitizing effect of a phenylbutyrate-derived histone deacetylase inhibitor in hepatocellular carcinoma, Int J Radiat Oncol Biol Phys. 2012 Jun. 1; 83(2); Zimmerman B, et. al., Efficacy of novel histone deacetylase inhibitor, AR42, in a mouse model of human T-lymphotropic virus type 1 adult T cell lymphoma, Leuk Res. 2011 November; 35(11):1491-7; Zhang S, et al., The novel histone deacetylase inhibitor, AR-42, inhibits gp130/Stat3 pathway and induces apoptosis and cell cycle arrest in multiple myeloma cells, Int J. Cancer. 2011 Jul. 1; 129(1):204-13.

The term “polymorph” or “polymorphic” refers to different crystalline forms of a chemical compound. Polymorphic forms of a compound may possess properties that affect the solubility, stability, bioavailability, and efficacy of a compound. Polymorphic forms of a compound can be compared, for example, to amorphous forms or other crystalline forms with respect to thermodynamic behaviors measured by a variety of techniques including, but not limited, to melting point, thermogravimetric analysis (TGA), differential scanning calorimetry (DSC), x-ray powder diffraction (XRPD), high performance liquid chromatography (HPLC), Raman microscopy, FT-IR spectroscopy, mass spectrometry (MS), and thermogravimetric analysis coupled with mass spectrometry (TG-MS). The physical stability of crystalline forms can be measured, for example, under conditions where the temperature and humidity in the environment are controlled for various time periods.

### SUMMARY

Aspects disclosed herein provide polymorphic or crystalline forms of AR-42, also known as (S)—N-hydroxy-4-(3-methyl-2-phenylbutanamido)benzamide having the following chemical structure:

[chemical structure]

In one aspect, the polymorphic forms include salts, solvates, hydrates, anhydrous, co-crystalline and other crystalline forms and combinations. The polymorphic forms can be formulated into a variety of dosage forms having increased stability, increased bioavailability, sustained release, and other properties. Polymorphic forms of AR-42 described herein are characterized by methods including X-ray powder diffraction patterns (XRPD), differential scanning calorimetry (DSC), and thermogravimetry mass spectrometry (TG-MS).

In another aspect, polymorphic forms of AR-42 can be made by combining AR-42 with any of the following exemplary solvents: methycyclohexane, isopentyl acetate, nethyl tetrahydrofuran-2, trimethylpentane 2,2,4-, diisopropyl ether, cumene, dichloroethane 1,2-, toluene, cyclohexanone, cyclohexane, anisole, diethyl carbonate (anyhydrous), octane, tert-butylmethyl ether (anhydrous), dimethoxyethane 1,2-, butyl acetate, absolute ethanol, dioxane, 1,4-, chloroform, methyl-1-propanol 2-, dimethyl-3-butanone 2,2-, hydroxy-4-methyl-2-pentanon 4-, tetrahydrofuran, fluorbenzene, chlorobenzene, acetonitrile, methanol, water, nitromethane, ethylbenzene, dimethyl-4-heptanone, 2,6-, trimethylbenzene 1,2,4-, dimethyl-3-butanone 2,2-, nitromethane, fluorobenzene, dioxane, 1,4, methyltetrahydrofuran, 2-, and pentyl acetate.

In one aspect, the AR-42 polymorphic forms can be made by mixing AR-42 or a salt thereof with any of the above solvents, or other suitable solvents, with or without heating of the mixture and subsequent cooling and or evaporation of the solvents at various rates in order to form precipitated material which can be analyzed as described herein.

### FIGURES

FIG. 1 shows exemplary XRPD patterns for different lots of Form A of AR-42;

FIG. 2 shows exemplary images of the AR-42 samples corresponding to the XRPD patterns of FIG. 1;

FIG. 3 shows exemplary XRPD patterns for the indicated polymorphic forms of AR-42;

FIG. 4 shows exemplary images of the AR-42 samples corresponding to the XRPD patterns of FIG. 3;

FIG. 5 shows exemplary XRPD patterns for the indicated polymorphic forms of AR-42;

FIG. 6 shows exemplary images of the AR-42 samples corresponding to the XRPD patterns of FIG. 5;

FIG. 7 shows exemplary XRPD patterns for the indicated polymorphic forms of AR-42;

FIG. 8 shows exemplary images of the AR-42 samples corresponding to the XRPD patterns of FIG. 7;

FIG. 9 shows exemplary XRPD patterns for the indicated polymorphic forms of AR-42;

FIG. 10 shows exemplary images of the AR-42 samples corresponding to the XRPD patterns of FIG. 9;

FIG. 11 shows exemplary XRPD patterns for the indicated polymorphic forms of AR-42;

FIG. 12 shows exemplary images of the AR-42 samples corresponding to the XRPD patterns of FIG. 11;

FIG. 13 shows an exemplary TG-MS of Form D2;

FIG. 14 shows an exemplary DSC of Form D2;

FIG. 15 shows an exemplary TG-MS of Form A_minus;

FIG. 16 shows an exemplary DSC of Form A_minus;

FIG. 17 shows an exemplary TG-MS of Form Z;

FIG. 18 shows an exemplary TG-MS of Form M1;

FIG. 19 shows an exemplary DSC of Form M1;

FIG. 20 shows an exemplary TG-MS of Form A_plus;

FIG. 21 shows an exemplary DSC of Form A_plus;

FIG. 22 shows an exemplary TG-MS of Form L2;

FIG. 23 shows an exemplary TG-MS of Form M2;

FIG. 24 shows an exemplary DSC of Form M2;

FIG. 25 shows an exemplary TG-MS of Form B1;

FIG. 26 shows an exemplary DSC of Form B1;

FIG. 27 shows an exemplary TG-MS of Form A5a;

FIG. 28 shows an exemplary DSC of Form A5a;

FIG. 29 shows an exemplary TG-MS of Form R;

FIG. 30 shows an exemplary DSC of Form R;

FIG. 31 shows an exemplary TG-MS of Form H2;

FIG. 32 shows an exemplary TG-MS of Form A5b;

FIG. 33 shows an exemplary DSC of Form A5b;

FIG. 34 shows an exemplary TG-MS of Form J1;

FIG. 35 shows an exemplary DSC of Form J1; and

FIG. 36 shows an exemplary DSC of Form L1.

### DETAILED DESCRIPTION

Before describing several exemplary aspects described herein, it is to be understood that the invention is not limited to the details of construction or process steps set forth in the following description. The aspects described herein are capable of being practiced or being carried out in various ways.

Aspects described herein provide polymorphic forms of AR-42 which have advantageous properties including but not limited to increased bioavailability, increased stability, and increased solubility. In one aspect, these properties relate to properties that will impart advantages with respect to formulating AR-42 into a suitable dosage form.

Polymorphic forms of AR-42 have varying physical and chemical properties with respect, for example, solubility, melting temperature, hygroscopy, and vapor pressure which may affect the stability of a particular dosage form of AR-42. Drug formulation and dosage form selection have a significant impact on the cost of manufacturing. Stability of a particular dosage form may also significantly impact the shelf life of the drug, required frequency of refills, and the cost of the drug to the patient. Thus, selecting a polymorphic form with desired chemical properties may affect the cost of manufacture, the effectiveness of the drug, and the cost and convenience of using the drug for the patient.

Physical properties such as flow, particle size, surface area, and hardness may significantly impact the pharmacokinetics of the drug. For example, the dissolution rate and half-life of the drug in the body will affect the maximum concentration in the blood, clearance of the drug, and whether the drug is resident in the body for the optimal period of time.

Polymorphic forms of AR-42 were identified by conducting solubility assessments in a variety of solvents (e.g., methycyclohexane, isopentyl acetate, nethyl tetrahydrofuran-2, trimethylpentane 2,2,4-, diisopropyl ether, cumene, dichloroethane 1,2-, toluene, cyclohexanone, cyclohexane, anisole, diethyl carbonate (anyhydrous), octane, tert-butylmethyl ether (anhydrous), dimethoxyethane 1,2-, butyl acetate, absolute ethanol, dioxane, 1,4-, chloroform, methyl-1-propanol 2-, dimethyl-3-butanone 2,2-, hydroxy-4-methyl-2-pentanon 4-, tetrahydrofuran, fluorbenzene, chlorobenzene, acetonitrile, methanol, water, nitromethane, ethylbenzene, dimethyl-4-heptanone, 2,6-, trimethylbenzene 1,2,4-, dimethyl-3-butanone 2,2-, nitromethane, fluorobenzene, dioxane, 1,4, methyltetrahydrofuran, 2-, and pentyl acetate). The resulting polymorphic forms of AR-42 were characterized by methods including XRPD, TG-MS, and DSC.

Methods for obtaining and characterizing polymorphic forms generally are known in the art as shown, for example, in H. G. Brittain, “Polymorphism in Pharmaceutical Solids”, 2nd edition [Informa Healthcare Press, New York, 2009], J. Bernstein, “Polymorphism in Molecular Crystals” [Clarendon Press, Oxford, 2002], and R. Hilfiker, “Polymorphism in the Pharmaceutical Industry [Wiley-VCH, Weinheim, 2006], incorporated by reference herein in their entirety.

FIG. 1 shows exemplary XRPD patterns for four lots of crystalline AR-42 starting material, termed Form A. Solubility was assessed in a set of 20 organic solvents. As shown in FIG. 1, the XRPD patterns are consistent across the four samples. HPLC purity is in the range of 97.89 to 99.48%.

FIG. 2 shows exemplary images of the AR-42 samples from 121 mL crystallization experiments using four different crystallization modes (cooling evaporative, anti-solvent addition, slurry, and solvent drop grinding.

In one aspect, AR-42 polymorphic form A8 has the XRPD pattern as shown in FIG. 3.

In one aspect, AR-42 polymorphic form A7 has the XRPD pattern as shown in FIG. 3.

In one aspect, AR-42 polymorphic form A6 has the XRPD pattern as shown in FIG. 3.

In one aspect, AR-42 polymorphic form A5b has the XRPD pattern as shown in FIG. 3.

In one aspect, AR-42 polymorphic form A5a has the XRPD pattern as shown in FIG. 3.

In one aspect, AR-42 polymorphic form A4 has the XRPD pattern as shown in FIG. 3.

In one aspect, AR-42 polymorphic form A3 has the XRPD pattern as shown in FIG. 3.

In one aspect, AR-42 polymorphic form A2 has the XRPD pattern as shown in FIG. 3.

In one aspect, AR-42 polymorphic form SM has the XRPD pattern as shown in FIG. 3.

In one aspect, AR-42 polymorphic form A_plus has the XRPD pattern as shown in FIG. 3.

In one aspect, AR-42 polymorphic form A_minus has the XRPD pattern as shown in FIG. 3.

In one aspect, AR-42 polymorphic form E has the XRPD pattern as shown in FIG. 5.

In one aspect, AR-42 polymorphic form D3 has the XRPD pattern as shown in FIG. 5.

In one aspect, AR-42 polymorphic form D2 has the XRPD pattern as shown in FIG. 5.

In one aspect, AR-42 polymorphic form D1 has the XRPD pattern as shown in FIG. 5.

In one aspect, AR-42 polymorphic form C has the XRPD pattern as shown in FIG. 5.

In one aspect, AR-42 polymorphic form B1 has the XRPD pattern as shown in FIG. 5.

In one aspect, AR-42 polymorphic form H2 has the XRPD pattern as shown in FIG. 7.

In one aspect, AR-42 polymorphic form H1 has the XRPD pattern as shown in FIG. 7.

In one aspect, AR-42 polymorphic form G_gel has the XRPD pattern as shown in FIG. 7.

In one aspect, AR-42 polymorphic form F5_wet has the XRPD pattern as shown in FIG. 7.

In one aspect, AR-42 polymorphic form F4_wet has the XRPD pattern as shown in FIG. 7.

In one aspect, AR-42 polymorphic form F3 has the XRPD pattern as shown in FIG. 7.

In one aspect, AR-42 polymorphic form F2 has the XRPD pattern as shown in FIG. 7.

In one aspect, AR-42 polymorphic form F1 has the XRPD pattern as shown in FIG. 7.

In one aspect, AR-42 polymorphic form J1 has the XRPD pattern as shown in FIG. 9.

In one aspect, AR-42 polymorphic form J2 has the XRPD pattern as shown in FIG. 9.

In one aspect, AR-42 polymorphic form K1 has the XRPD pattern as shown in FIG. 9.

In one aspect, AR-42 polymorphic form L1 has the XRPD pattern as shown in FIG. 9.

In one aspect, AR-42 polymorphic form L2 has the XRPD pattern as shown in FIG. 9.

In one aspect, AR-42 polymorphic form M1 has the XRPD pattern as shown in FIG. 9.

In one aspect, AR-42 polymorphic form M2 has the XRPD pattern as shown in FIG. 9.

In one aspect, AR-42 polymorphic form R has the XRPD pattern as shown in FIG. 11.

In one aspect, AR-42 polymorphic form S has the XRPD pattern as shown in FIG. 11.

In one aspect, AR-42 polymorphic form T_wet has the XRPD pattern as shown in FIG. 11.

In one aspect, AR-42 polymorphic form X_gel has the XRPD pattern as shown in FIG. 11.

In one aspect, AR-42 polymorphic form Z has the XRPD pattern as shown in FIG. 11.

Solubility assessment experiments were carried out in a variety of solvents followed by storage in a climate chamber for 48 to 72 hours at 40 degrees centigrade and 75% relative humidity. In another aspect, Form A_minus, Form A_plus, Form A5a, Form A5b, Form D2, Form B1, Form J1, Form J2, Form M1, Form M2, Form L1, Form L2, and Form Z exhibited a particular degree of stability and were examined using DSC and TG-MS as shown in FIGS. 13 and 14 (Form D2), FIGS. 15 and 16 (Form A_minus), FIG. 17 (Form Z), FIGS. 18 and 19 (Form M1), FIGS. 20 and 21 (Form A_plus), FIG. 22 (L2), FIGS. 23 and 24 (Form M2); FIGS. 25 and 26 (Form B1); FIGS. 27 and 28 (Form A5a); FIGS. 29 and 30 (Form R); FIG. 31 (Form H2); FIGS. 32 and 33 (Form A5b; FIGS. 34 and 35 (Form J1); and FIG. 36 (Form L1).

In another aspect, the XRPD patterns for Form J1, Form A5a, Form M1 and Form M2, remained unchanged following storage exposure in the climate chamber in a climate chamber for 48 to 72 hours at 40 degrees centigrade and 75% relative humidity. In this aspect, these forms are considered particularly stable.

In another aspect, using process solvents used to produce AR-42 Form A (i.e. water or ethanol) yielded Form D2, Form H1, and Form M2. In yet another aspect, the TG-MS analysis of Form D2 indicates it may be solvated (e.g., FIG. 13). In another aspect, the TG-MS and DSC analysis indicates that Form A_minus, Form A_plus, Form M1, Form M2, Form A5a, Form A5b and Form J1 are anhydrates. In yet another aspect, Form M2 is a potential anhydrate form, with somewhat lower melting point (i.e. Tₚₑₐₖ=187.4° C.) as compared to starting material Form A (i.e. Tₚₑₐₖ=204.2° C.).

The AR-42 polymorphic or crystalline forms can be used to treat a patient in need of treatment as described herein. The terms “treat,” “prevent,” or similar terms, as used herein, do not necessarily mean 100% or complete treatment or prevention. Rather, these terms refer to various degrees of treatment or prevention of a particular disease (e.g., 100%, 90%, 80%, 70%, 60%, 50%, 40%, 30%, 20%, 10%, 5%, or 1%) as recognized in the art as being beneficial. The terms “treatment” or “prevention” also refer to delaying onset of a disease for a period of time or delaying onset indefinitely. The term “treatment” or “treating” refers to administering a drug or treatment to a patient or prescribing a drug to a patient where the patient or a third party (e.g., caretaker, family member, or health care professional) administers the drug or treatment.

The AR-42 polymorphic or crystalline forms also encompass derivatives. In one embodiment, the term “derivative” includes, but is not limited to, ether derivatives, acid derivatives, amide derivatives, ester derivatives and the like. Methods of preparing these derivatives are known to a person skilled in the art. For example, ether derivatives are prepared by the coupling of the corresponding alcohols. Amide and ester derivatives are prepared from the corresponding carboxylic acid by a reaction with amines and alcohols, respectively.

The AR-42 polymorphic or crystalline forms also encompass hydrates of AR-42 polymorphic or crystalline forms (e.g., hemihydrate, monohydrate, dihydrate, trihydrate and the like). Hydrates of AR-42 may be prepared by contacting AR-42 with water under suitable conditions to produce the hydrate of choice.

The AR-42 polymorphic or crystalline forms also encompass metabolites of AR-42 polymorphic or crystalline forms. “Metabolite” or “metabolites” refer to any substance produced from another substance by metabolism or a through a metabolic process of a living cell or organ.

Any of the polymorphic AR-42 forms described herein can be administered orally, parenterally (IV, IM, depot-IM, SQ, and depot-SQ), sublingually, intranasally (inhalation), intrathecally, topically, or rectally. Dosage forms known to those of skill in the art are suitable for delivery of the AR-42 polymorphic forms described herein.

The AR-42 polymorphic compounds can be formulated into suitable pharmaceutical preparations such as tablets, capsules, or elixirs for oral administration or in sterile solutions or suspensions for parenteral administration. The AR-42 polymorphic compounds described herein can be formulated into pharmaceutical compositions using techniques and procedures well known in the art.

In one aspect, about 0.1 to 1000 mg, about 5 to about 100 mg, or about 10 to about 50 mg of the AR-42 polymorphic compounds, or a physiologically acceptable salt or ester can be compounded with a physiologically acceptable vehicle, carrier, excipient, binder, preservative, stabilizer, flavor, etc., in a unit dosage form as called for by accepted pharmaceutical practice. The amount of active substance in compositions or preparations comprising the AR-42 polymorphic compounds is such that a suitable dosage in the range indicated is obtained.

In another aspect, the compositions can be formulated in a unit dosage form, each dosage containing from about 1 to about 500 mg, or about 10 to about 100 mg of the active ingredient. The term “unit dosage from” refers to physically discrete units suitable as unitary dosages for human subjects and other mammals, each unit containing a predetermined quantity of active material calculated to produce the desired therapeutic effect, in association with a suitable pharmaceutical excipient.

In one aspect, one or more of the AR-42 polymorphic compounds are mixed with a suitable pharmaceutically acceptable carrier to form compositions. Upon mixing or addition of the compound(s), the resulting mixture may be a solution, suspension, emulsion, or the like. Liposomal suspensions may also be used as pharmaceutically acceptable carriers. These may be prepared according to methods known to those skilled in the art. The form of the resulting mixture depends upon a number of factors, including the intended mode of administration and the solubility of the compound in the selected carrier or vehicle. In one aspect, the effective concentration is sufficient for lessening or ameliorating at least one symptom of the disease, disorder, or condition treated and may be empirically determined.

Pharmaceutical carriers or vehicles suitable for administration of the AR-42 polymorphic compounds described herein include any such carriers suitable for the particular mode of administration. In addition, the active materials can also be mixed with other active materials that do not impair the desired action, or with materials that supplement the desired action, or have another action. The compounds may be formulated as the sole pharmaceutically active ingredient in the composition or may be combined with other active ingredients.

In another aspect, if the AR-42 polymorphic compounds exhibit insufficient solubility, methods for solubilizing may be used. Such methods are known and include, but are not limited to, using co-solvents such as dimethylsulfoxide (DMSO), using surfactants such as TWEEN, and dissolution in aqueous sodium bicarbonate. Derivatives of the compounds, such as salts or prodrugs, may also be used in formulating effective pharmaceutical compositions.

The concentration of the compound is effective for delivery of an amount upon administration that lessens or ameliorates at least one symptom of the disorder for which the compound is administered. Typically, the compositions are formulated for single dosage administration.

In another aspect, the AR-42 polymorphic compounds described herein may be prepared with carriers that protect them against rapid elimination from the body, such as time-release formulations or coatings. Such carriers include controlled release formulations, such as, but not limited to, microencapsulated delivery systems. The active compound can be included in the pharmaceutically acceptable carrier in an amount sufficient to exert a therapeutically useful effect in the absence of undesirable side effects on the patient treated. The therapeutically effective concentration may be determined empirically by testing the compounds in known in vitro and in vivo model systems for the treated disorder.

In another aspect, the AR-42 polymorphic compounds and compositions described herein can be enclosed in multiple or single dose containers. The enclosed compounds and compositions can be provided in kits, for example, including component parts that can be assembled for use. For example, an AR-42 polymorphic compound in lyophilized form and a suitable diluent may be provided as separated components for combination prior to use. A kit may include AR-42 polymorphic compound and a second therapeutic agent for co-administration. The AR-42 polymorphic compound and second therapeutic agent may be provided as separate component parts. A kit may include a plurality of containers, each container holding one or more unit dose of the AR-42 polymorphic compounds described herein. In one aspect, the containers can be adapted for the desired mode of administration, including, but not limited to tablets, gel capsules, sustained-release capsules, and the like for oral administration; depot products, pre-filled syringes, ampoules, vials, and the like for parenteral administration; and patches, medipads, creams, and the like for topical administration.

The concentration of the AR-42 polymorphic compound in the pharmaceutical composition will depend on absorption, inactivation, and excretion rates of the active compound, the dosage schedule, and amount administered as well as other factors known to those of skill in the art.

In another aspect, the active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

If oral administration is desired, the compound can be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition.

The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a glidant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

The active materials can also be mixed with other active materials that do not impair the desired action, or with materials that supplement the desired action. The AR-42 polymorphic compounds can be used, for example, in combination with an antitumor agent, a hormone, a steroid, or a retinoid. The antitumor agent may be one of numerous chemotherapy agents such as an alkylating agent, an antimetabolite, a hormonal agent, an antibiotic, colchicine, a vinca alkaloid, L-asparaginase, procarbazine, hydroxyurea, mitotane, nitrosoureas or an imidazole carboxamide. Suitable agents include those agents which promote depolarization of tubulin. Examples include colchicine and vinca alkaloids, including vinblastine and vincristine.

In another aspect, the AR-42 polymorphic forms described herein can be co-administered or administered before or after immunization of a patient with a vaccine to enhance the immune response to the vaccine. In one aspect the vaccine is a DNA vaccine, for example, and HPV vaccine.

In one aspect, solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerin, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include, but are not limited to, physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers. These may be prepared according to methods known in the art.

In another aspect, the AR-42 polymorphic compounds may be prepared with carriers that protect the compound against rapid elimination from the body, such as time-release formulations or coatings. Such carriers include controlled release formulations, such as, but not limited to, implants and microencapsulated delivery systems, and biodegradable, biocompatible polymers such as collagen, ethylene vinyl acetate, polyanhydrides, polyglycolic acid, polyorthoesters, polylactic acid, and the like. Methods for preparation of such formulations are known to those skilled in the art.

In yet another aspect, compounds employed in the methods of the disclosure may be administered enterally or parenterally. When administered orally, compounds employed in the methods of the disclosure can be administered in usual dosage forms for oral administration as is well known to those skilled in the art. These dosage forms include the usual solid unit dosage forms of tablets and capsules as well as liquid dosage forms such as solutions, suspensions, and elixirs. When the solid dosage forms are used, they can be of the sustained release type so that the compounds employed in the methods described herein need to be administered only once or twice daily.

The oral dosage forms can be administered to the patient 1, 2, 3, or 4 times daily. The AR-42 polymorphic compounds described herein can be administered either three or fewer times, or even once or twice daily. Hence, the HDAC inhibitor compounds employed in the methods of the disclosure be administered in oral dosage form. Whatever oral dosage form is used, they can be designed so as to protect the compounds employed in the methods described herein from the acidic environment of the stomach. Enteric coated tablets are well known to those skilled in the art. In addition, capsules filled with small spheres each coated to protect from the acidic stomach, are also well known to those skilled in the art.

The terms “therapeutically effective amount” and “therapeutically effective period of time” are used to denote treatments at dosages and for periods of time effective to reduce neoplastic cell growth. As noted above, such administration can be parenteral, oral, sublingual, transdermal, topical, intranasal, or intrarectal. In one aspect, when administered systemically, the therapeutic composition can be administered at a sufficient dosage to attain a blood level of the compounds of from about 0.1 μM to about 100 mM. For localized administration, much lower concentrations than this can be effective, and much higher concentrations may be tolerated. One of skill in the art will appreciate that such therapeutic effect resulting in a lower effective concentration of the AR-42 polymorphic compound may vary considerably depending on the tissue, organ, or the particular animal or patient to be treated. It is also understood that while a patient may be started at one dose, that dose may be varied overtime as the patient's condition changes.

It should be apparent to one skilled in the art that the exact dosage and frequency of administration will depend on the particular compounds employed in the methods of the disclosure administered, the particular condition being treated, the severity of the condition being treated, the age, weight, general physical condition of the particular patient, and other medication the individual may be taking as is well known to administering physicians who are skilled in this art.

### EXAMPLES

### Example 1

### Solubility Assessment Experiments

Tables 1-2 illustrate solubility experiments conducted on the indicated Form as classified by XRPD patterns.

TABLE 1

| | | | | | Concen- |
|---|---|---|---|---|---|
| | | XRPD | | | tration |
| Index | Well | Form | Exp. ID | Solvent | (mg/mL) |
| 1 | 995: B5 | n | SAS33 | Trifluoroethanol, 2, 2, | 7 |
| | | | | 2- | |
| 2 | 995: G5 | Form | SAS38 | Hexafluorobenzene | <1 |
| | | A_wet** | | | |
| 3 | 995: F5 | Form | SAS37 | Amyl Ether | <1 |
| | | A + Am* | | | |
| 4 | 995: A5 | Form B1 | SAS32 | Dimethyl-4- | 2 |
| | | | | heptanone 2,6- | |
| 5 | 955: E5 | n | SAS36 | Toluene | <1 |
| 6 | 995: C5 | n | SAS34 | Chloroform | <2 |
| 7 | 995: D5 | n | SAS35 | Nitromethane | 1 |
| 8 | 996: B4 | Am_oily | SAS1 | NMP | 482.5 |

n - low yield material therefore the form could not be assigned.
*(Form A + Am) - crystalline Form A is present as well as an amorphous phase: the amorphous could be from a solid amorphous material as well as from a slightly wet material.
**wet solid material.

TABLE 2

| Index | Well | XRPD Form | Exp. ID | Concentration (mg/mL) | Solvent |
|---|---|---|---|---|---|
| 1 | 997: C3 | Form A | SAS5 | 20.3 | Dimethyl-4-heptanone 2,6- |
| 2 | 997: D3 | Form A | SAS6 | 20.6 | Trifluoroethanol, 2,2,2- |
| 3 | 997: I3 | Form A | SAS11 | 21.2 | Nitromethane |
| 4 | 997: L3 | Form A | SAS14 | 21.4 | Hexafluorobenzene |
| 5 | 997: K3 | Form A | SAS13 | 18.4 | Amyl Ether |
| 6 | 997: J3 | Form A | SAS12 | 18.8 | Toluene |
| 7 | 997: G3 | Form A + Form B1 | SAS9 | 108.9 | Acetone |
| 8 | 997: E3 | Form B1 | SAS7 | 20.8 | Chloroform |
| 9 | 997: F3 | Form B1_wet** | SAS8 | 243.7 | Isophorone |
| 10 | 997: A4 | Form B1 + Form D1 | SAS15 | 32.3 | Dioxane, 1,4- |
| 11 | 997: A3 | Form C | SAS2 | 472.5 | Dimethyl Sulfoxide |
| 12 | 997: B3 | Form D1 | SAS4 | 19.7 | Nitrobenzene |
| 13 | 997: H3 | Form E | SAS10 | 313.3 | Tetrahydrofuran |

**wet solid material.

### Example 2

### Anti-Solvent Addition Crystallization Experiments

Tables 3-5 illustrate anti-solvent addition crystallization experiments conducted on the indicated Form. In this example, saturated solutions of AR42 were prepared in the following solvents: Acetone, 2-Butanone, Methanol, Ethanol, Isopropanol, Acetonitrile, 1,4-Dioxane and Dimethylformamide. Excess AR42 was added to the solvent and the suspension equilibrated for 24 hours. After equilibration, the suspension was filtered through a 0.2 μM filter to remove particulate solids.

An equal volume of anti-solvent selected from the following list was added to 300 μL of the saturated solutions of AR42: Toluene, Nitrobenzene, Nitromethane, Water, n-Heptane, Cyclohexane. An additional 300 μL of anti-solvent was added if no precipitation occurred. Aliquots of 300 μL of anti-solvent were added until precipitation occurred or the total volume was 1,200 μL of anti-solvent. The solution was allowed to incubate for 60 minutes at room temperature in between the addition of anti-solvent. If no precipitation occurred after the last addition of anti-solvents, samples were incubated for 24 hours at 4° C. Samples in which precipitation occurred were centrifuged for 10 minutes at 3000 rpm to separate the liquid from the solid. The liquid phase was removed and the solid phase dried and analyzed by XRPD. The solutions in which no precipitation occurred were placed under deep vacuum and dried to completion. Solids were collected and analysed by XRPD.

The following combinations of solvent and anti-solvent were used:

Acetone with Toluene or Nitrobenzene or Nitromethane or water;

2-Butanone with Toluene or n-Heptane or Cyclohexane or Nitrobenzene or Nitromethane;

Methanol with Toluene or Nitrobenzene or Nitromethane or water;

Ethanol with Toluene or Nitrobenzene or Nitromethane or water;

Isopropanol with Toluene or n-Heptane or Cyclohexane or Nitrobenzene or Nitromethane or water;

Acetonitrile with Toluene or Nitrobenzene or Nitromethane or water;

1,4-Dioxane with Toluene or Cyclohexane or Nitrobenzene or Nitromethane or water; and

Dimethylformamide with Toluene or Nitrobenzene or Nitromethane or water.

TABLE 3

| | | | | | Anti solvent - | | | |
|---|---|---|---|---|---|---|---|---|
| | Well before | | | | Solvent | Exp. | Concentration | |
| Index | stability | XRPD Form^{a} | XRPD Form^{b} | Anti solvent | Ratio (x:1) | ID | (mg/mL) | Stock Solvent |
| 1 | AS01:E2 | Am_oily | n | Water | 2 | AS36 | 335 | Dimethylformamide |
| | | | | | | | | N,N- |
| 2 | AS01: C1 | Form A | Form A | Nitromethane | 1 | AS3 | 175.9 | Acetone |
| 3 | AS01:J1 | Form A | Form A | Water | 1 | AS13 | 390.5 | methanol |
| 4 | AS01:D1 | Form A | Form A | Water | 2 | AS4 | 175.9 | Acetone |
| 5 | AS01:F1 | Form A_minus | Form A_minus | Heptane, N- | 1 | AS6 | 130.7 | Butanone 2- |
| 6 | AS01:B2 | Form A_wet** | Form A + Am* | Water | 2 | AS23 | 227.8 | Propanol 2- |
| 7 | AS01:K1 | Form A + | Form A | Water | 1 | AS17 | 134.1 | Ethanol |
| | | Form H1 | | | | | | |
| 8 | AS01:L1 | Form A2 | Form A | Heptane, N- | 4 | AS19 | 227.8 | Propanol 2- |
| 9 | AS01:I1 | Form B1 + | Form A | Nitromethane | 1 | AS9 | 130.7 | Butanone 2- |
| | | Form F5 | | | | | | |
| 10 | AS01:B1 | Form D1_wet** | Form D2 | Nitrobenzene | 1 | AS2 | 175.9 | Acetone |
| 11 | AS01:A1 | Form F1 | Form A | Toluene | 1 | AS1 | 175.9 | Acetone |
| 12 | AS01:E1 | Form F1 | Form A + Am* | Toluene | 1 | AS5 | 130.7 | Butanone 2- |
| 13 | AS01:D2 | Form G_gel | Form A + Am* | Cyclohexane | 2 | AS29 | 46.8 | Dioxane, 1,4- |
| 14 | AS01:G1 | Form W + Am* | Form A + Form | Cyclohexane | 1 | AS7 | 130.7 | Butanone 2- |
| 15 | AS01:C2 | Wet sample | Form A + Am* | Toluene | 3 | AS28 | 46.8 | Dioxane, 1,4- |
| 16 | AS01:H1 | Wet sample | Form W + Am* | Nitrobenzene | 1 | AS3 | 130.7 | Butanone 2- |
| 17 | AS01:A2 | n | n | Cyclohexane | 3 | AS20 | 227.8 | Propanol 2- |

^{a}XRPD form assigned after Recrystallization (as wet or dry solid) thus before the stability studies;
^{b}XRPD form assigned after the stability studies (48-72 H, at 40° C./75% RH).
*the assigned crystalline form is present as well as an amorphous phase; the amorphous could be from a solid amorphous material as well as from a slightly wet material.
**wet solid material: n- low yield material therefore the form could not be assigned.

TABLE 4

| | | | | AS - Solvent | Exp. | Concentration | |
|---|---|---|---|---|---|---|---|
| Index | Well | XRPD Form^{a} | Anti solvent | Ratio (x:1) | ID | (mg/mL) | Stock Solvent |
| 1 | AS02:E1 | Form E + Form I | Nitromethane | 3 | AS26 | 13.5 | Acetonitrile |
| 2 | AS02:A1 | Form F5_wet** | Nitromethane | 4 | AS12 | 390.5 | methanol |
| 3 | AS02:C1 | Form H1 | Nitromethane | 3 | AS16 | 134.1 | Ethanol |
| 4 | AS02:B1 | Form H1_wet** | Toluene | 3 | AS14 | 134.1 | Ethanol |
| 5 | AS02:D1 | Wet sample | Toluene | 3 | AS18 | 227.8 | Propanol 2- |

^{a}XRPD form assigned after Recrystallization (as wet or dry solid) thus before the stability studies;
**wet solid material.

TABLE 5

| | Well before | | | | AS- Solvent | Exp. | Concentration | |
|---|---|---|---|---|---|---|---|---|
| Index | stability | XRPD Form^{a} | XRPD Form^{b} | Anti solvent | Ratio (x:1) | ID | (mg/mL) | Stock Solvent |
| 1 | AS04:B4 | Am_oily | Form A | Nitromethane | 4 | AS35 | 335 | Dimethylformamide |
| | | | | | | | | N,N- |
| 2 | AS04:H3 | Form B1 + | Form A_low | Water | 4 | AS27 | 13.5 | Acetonitrile |
| | | Form F1 | yield | | | | | |
| 3 | AS04:D3 | Form D1 | Form D2 + Am* | Nitrobenzene | 4 | AS21 | 227.8 | Propanol 2- |
| 4 | AS04:I3 | Form D1 | Form D2 | Nitrobenzene | 4 | AS30 | 46.8 | Dioxane, 1,4- |
| 5 | AS04:G3 | Form D1 | Form D2 + Am* | Nitrobenzene | 4 | AS25 | 13.5 | Acetonitrile |
| 6 | AS04:B3 | Form D1 | Form D2 | Nitrobenzene | 4 | AS11 | 390.5 | methanol |
| 7 | AS04:A4 | Form D1 | Form D2 | Nitrobenzene | 4 | AS34 | 335 | Dimethylformamide |
| | | | | | | | | N,N- |
| 8 | AS04:C3 | Form D1 | Form D2 | Nitrobenzene | 4 | AS15 | 134.1 | Ethanol |
| 9 | AS04:K3 | Form D1 + | Form A + | Water | 4 | AS32 | 46.8 | Dioxane, 1,4- |
| | | Form F1 | Form B1 | | | | | |
| 10 | AS04:J3 | Form D1 + | Form A + | Nitromethane | 4 | AS31 | 46.8 | Dioxane, 1,4- |
| | | Form F1 | Form D3_min*** | | | | | |
| 11 | AS04:A3 | Form D3 | Form A + | Toluene | 4 | AS10 | 390.5 | methanol |
| | | | Form D3 | | | | | |
| 12 | AS04:E3 | Form F1 | Form A | Nitromethane | 4 | AS22 | 227.8 | Propanol 2- |
| 13 | AS04:F3 | Form F1 | Form A | Toluene | 4 | AS24 | 13.5 | Acetonitrile |
| 14 | AS04:L3 | Form T_wet** | Form A | Toluene | 4 | AS33 | 335 | Dimethylformamide |
| | | | | | | | | N,N- |

*the assigned crystalline form is present as well as an amorphous phase; the amorphous could be from a solid amorphous material as well as from a slightly wet material.
**wet solid material.
***a minimum amount of crystalline form is present in mixture with a predominant crystalline form.

### Example 3

### Cooling-Evaporative Crystallization Experiments

Tables 6-7 illustrate cooling evaporative crystallization experiments conducted on the indicated Form. Slurries of AR42 were prepared by adding an excess of AR42 to standard HPLC vials containing the following solvents and solvent mixtures: Methanol, Tetrahydrofuran, Ethyl acetate, 2-Methyltetrahydrofuran, Ethanol, Acetonitrile, 1,2-Dichloroethane, Fluorbenzene, 1,2-Dimethoxyethane, Propionitrile, Isobutanol, Isopropyl acetate, Butyl Acetate, Chlorobenzene, 2-Ethoxyethanol, 1-Pentanol, Cyclohexanone, 4-Hydroxy-4-Methyl-2-Pentanone, Methanol/Acetonitrile (50:50), Acetonitrile/Chloroform (50:50), Methanol/p-Xylene (50:50), 1,2-Dimethoxyethane/Methanol (50:50), 1,2-Dimethoxyethane/Isopropylether (50:50), Formamide/Methanol (50:50), Cyclohexanone/Tetrahydrofuran (50:50), Cyclohexane/N-methyl-2-pyrrolidone (50:50), Cyclohexane/Cyclohexanone (50:50), Methylcyclohexane/1,2-Dichloroethane (50:50), Cyclohexanone/1,4-Dioxane (50:50), Octane/1-Octanol (50:50), Xylene-p/Nitrobenzene (50:50), n-Heptane/1-Octanol (50:50), Cyclohexanone//Methylcyclohexane (50:50), Methylcyclohexane/4-Hydroxy-4-methyl-2-pentanone (50:50), Anisole/Nitrobenzene (50:50) and Cyclohexanone/N-methyl-2-pyrrolidone (50:50).

The vials were placed in the Crystal16, heated to 50° C. and held at that temperature for 1 hour. Subsequently the vials were cooled to 5° C. with a cooling rate of 10° C./h. The samples were aged at 5° C. for 72 hours.

Samples in which solids were present were centrifuged for 10 minutes at 3000 rpm to separate the liquid from the solid. The liquid phase was removed and the solid phase dried and analysed by XRPD. The samples in which no solids were present were placed under deep vacuum and dried to completion. Solids were collected and analysed by XRPD.

TABLE 6

| | Well after | | | | | | | | | Stock |
|---|---|---|---|---|---|---|---|---|---|---|
| | recrystal- | | | | Exp. | Solvent | Solvent | Solvent | Solvent | Conc. |
| Index | lization | XRPD Form^{a} | XRPD Form^{b} | XRPD Form^{c} | ID | 1 (%) | 2 (%) | 1 | 2 | (mg/mL) |
| 1 | C01:E2 | Form A | Form A | Form A_wet** | ML26 | 50 | 50 | Cyclohexane | N-methyl-2- | 37 |
| | | | | | | | | | pyrrolidone | |
| 2 | C01:B2 | Form A | Form A | Form A | ML23 | 50 | 50 | Dimethoxyethane | Isopropyl | 31 |
| | | | | | | | | 1,2- | ether | |
| 3 | C01:G1 | Form A | Form A + | Form A_wet** | ML10 | 100 | — | Propionitrile | — | 31 |
| | | | Form B1 | | | | | | | |
| 4 | C01:E1 | Form A_wet** | Form A | Form A_wet** | ML7 | 100 | — | Dichloroethane | — | 76 |
| | | | | | | | | 1,2- | | |
| 5 | C01:H2 | Form A_wet | Form U1 + Am* | Form Z | ML32 | 50 | 50 | Heptane, N- | Octanol-1 | 34 |
| 6 | C01:F1 | Form A_wet** | Form A_wet** | Form A_wet** | ML8 | 100 | — | Fluorbenzene | — | 28 |
| 7 | C01:A1 | Form A_wet** | Form A | Form A + Am* | ML3 | 100 | — | Ethyl acetate | — | 34 |
| 8 | C01:J1 | Form A + Am* | Form U1 + Am* | Form A + Am* | ML13 | 100 | — | Butyl Acetate | — | 37 |
| 9 | C01:K1 | Form A + Am | Form A_wet** | Form A + Am* | ML14 | 100 | — | Chlorobenzene | — | 42 |
| 10 | C01:H1 | Form A + | Form B1_wet** | Form A_wet** | ML11 | 100 | — | Isobutanol | — | 132 |
| | | Form B1 | | | | | | | | |
| 11 | C01:D1 | Form A + | Form A + | Form A + | ML6 | 100 | — | Acetonitrile | — | 32 |
| | | Form J1 | Form J1 | Form J1 | | | | | | |
| 12 | C01:F2 | Form A2 + Am | Form U1 + Am | Form Z_wet** | ML30 | 50 | 50 | Octane | Octanol-1 | 34 |
| 13 | C01:A2 | Form B1_wet** | Form A7 + Am* | Form A | ML20 | 50 | 50 | Acetonitrile | Chloroform | 37 |
| 14 | C01:I1 | Form B1_wet** | Form B1_wet** | Form A_wet** | ML12 | 100 | — | Isopropyl | — | 36 |
| | | | | | | | | acetate | | |
| 15 | C01:G2 | Form D1_wet** | Form D1_wet** | Form A_wet** | ML31 | 50 | 50 | Xylene-p | Nitrobenzene | 36 |
| 16 | C01:C1 | Form H1 | Form A | Form A | ML5 | 100 | — | Ethanol | — | 133 |
| 17 | C01:B1 | Form K_wet | Form A | Form A | ML4 | 100 | — | Methyltetra- | — | 79 |
| | | | | | | | | hydrofuran, 2- | | |
| 18 | C01:C2 | Form L1 | Form L1 | Form L1 | ML24 | 50 | 50 | Formamide | methanol | 103 |
| 19 | C01:D2 | Form X_gel | Form U2 + Am* | Form A_wet** | ML27 | 50 | 50 | Cyclohexane | Cyclohexanone | 36 |
| 20 | C01:L1 | Form Z_wet | Form U1 + Am* | Form Z_wet** | ML16 | 100 | — | Pentanol 1- | — | 132 |
| 21 | C01:I2 | Wet sample | Form D1_wet** | Form A | ML36 | 50 | 50 | Cyclohexanone | N-methyl-2- | 111 |
| | | | | | | | | | pyrrolidone | |

*the assigned crystalline form is present as well as an amorphous phase; the amorphous could be from a solid amorphous material as well as from a slightly wet material.
**wet solid material.
***a minimum amount of crystalline form is present in mixture with a predominant crystalline form.
^{a}XRPD form assigned after Recrystallization (as wet or dry solid) thus before the stability studies;
^{b}XRPD form assigned after storage of the samples under ambient conditions for 1-1.5 weeks.
^{c}XRPD form assigned after the stability studies (48-72 H, at 40° C./75% RH).

TABLE 7

| | Well after | | | | | | | | | Stock |
|---|---|---|---|---|---|---|---|---|---|---|
| | recrystal- | | | | Exp. | Solvent | Solvent | Solvent | Solvent | Conc. |
| Index | lization | XRPD Form^{a} | XRPD Form^{b} | XRPD Form^{c} | ID | 1 (%) | 2 (%) | 1 | 2 | (mg/mL) |
| 1 | C02:J1 | Form A | Form A | Form A | ML10 | — | — | Propionitrile | — | 31 |
| 2 | C02:C1 | Form A | Form A | Form A | ML3 | — | — | Ethyl acetate | — | 34 |
| 3 | C02:D3 | Form A | Form A | Form A | ML28 | 50 | 50 | Methyl- | Dichloro- | 40 |
| | | | | | | | | cyclohexane | ethane 1,2- | |
| 4 | C02:F1 | Form A + | Form A + | Form J1 | ML6 | — | — | Aceto- | — | 32 |
| | | Form J1 | Form J1 | | | | | nitrile | | |
| 5 | C02:K1 | Form A + | Form A + | Form A_low | ML11 | — | — | Isobutanol | — | 132 |
| | | Form J1 | Form J1 | yield | | | | | | |
| 5 | C02:K2 | Form A3 | Form L2 | Form L2 | ML23 | 50 | 50 | Dimethoxy- | Isopropyl | 31 |
| | | | | | | | | ethane 1,2- | ether | |
| 7 | C02:A2 | Form A3 | Form A + | Form A | ML13 | — | — | Butyl | — | 37 |
| | | | Form A3 | | | | | Acetate | | |
| 8 | C02:E1 | Form A4 | Form A + | Form A | ML5 | — | — | Ethanol | — | 133 |
| | | | Form A3 | | | | | | | |
| 9 | C02:A3 | Form A4 | Form R | Form A | ML25 | 50 | 50 | Cyclo- | Tetra- | 107 |
| | | | | | | | | hexanone | hydrofuran | |
| 10 | C02:C3 | Form A4 | Form B1_wet** | Form A + | ML27 | 50 | 50 | Cyclohexane | Cyclo- | 36 |
| | | | | Form B1 | | | | | hexanone | |
| 11 | C02:H2 | Form A7 | Form A7 | Form A_plus | ML20 | 50 | 50 | Acetonitrile | Chloroform | 37 |
| 12 | C02:D1 | Form A8 | Form A8 | Form A | ML4 | — | — | Methyltetra- | — | 79 |
| | | | | | | | | hydrofuran, 2- | | |
| 13 | C02:G3 | Form B1 | Form B1 | Form B1 | ML33 | 50 | 50 | Cyclo- | Methyl- | 37 |
| | | | | | | | | hexanone | cyclohexane | |
| 14 | C02:F2 | Form B1 | Form B1 | A + | ML18 | — | — | [symbol] | — | 144 |
| | | | | Form B1 | | | | pentanon 4- | | |
| 15 | C02:D2 | Form B1 | Form B1 | Form A + | ML16 | — | — | Pentanol 1- | — | 132 |
| | | | | Form B1 | | | | | | |
| 16 | C02:E3 | Form B1 | Form B1 | Form A + | ML29 | 50 | 50 | Cyclo- | Dioxane, 1,4- | 81 |
| | | | | Form B1 | | | | hexanone | | |
| 17 | C02:H3 | Form B1_wet** | Form B1_wet** | Form A + | ML34 | 50 | 50 | Methyl- | Hydroxy-4- | 28 |
| | | | | Form B1 | | | | cyclohexane | methyl-2- | |
| | | | | | | | | | pentanon 4- | |
| 18 | C02:I3 | Form D1 | Form D1 | Form A | ML35 | 50 | 50 | Anisole | Nitrobenzene | 36 |
| 19 | C02:F3 | Form D1_wet | Form D1_wet | Form A + Am* | ML31 | 50 | 50 | Xylene-p | Nitrobenzene | 36 |
| 20 | C02:H1 | Form F1 | Form A + | Form A | ML8 | — | — | Fluorbenzene | — | 28 |
| | | | Form F1 | | | | | | | |
| 21 | C02:G1 | Form F2 | Form A3 | Form A | ML7 | — | — | Dichloro- | — | 76 |
| | | | | | | | | ethane 1,2- | | |
| 22 | C02:B2 | Form F3 | Form A + | Form A | ML14 | — | — | Chlorobenzene | — | 42 |
| | | | Form F1 | | | | | | | |
| 23 | C02:B1 | Form J1 | Form J1 | Form J1 | ML2 | — | — | Tetra- | — | 383 |
| | | | | | | | | hydrofuran | | |
| 24 | C02:J2 | Form J1 | Form J2 | Form J2 | ML22 | 50 | 50 | Dimethoxy- | methanol | 94 |
| | | | | | | | | ethane 1,2- | | |
| 25 | C02:I1 | Form J1 | Form J2 | Form J1 | ML9 | — | — | Dimethoxy- | — | 315 |
| | | | | | | | | ethane 1,2- | | |
| 26 | C02:G2 | Form J1 | Form B1 + | Form A + | ML19 | 50 | 50 | methanol | Aceto- | 97 |
| | | | Form J1 | Form J1 | | | | | nitrile | |
| 27 | C02:L1 | Form L1 | Form L1 | Form L1 | ML12 | — | — | Isopropyl | — | 36 |
| | | | | | | | | acetate | | |
| 28 | C02:L2 | Form L1 | Form L1 | Form L1 | ML24 | 50 | 50 | Formamide | methanol | 103 |
| 29 | C02:A1 | Form M1 | Form M1 | Form M1 | ML1 | — | — | methanol | — | 539 |
| 30 | C02:C2 | Form M2 | Form M2 | Form M2 | ML15 | — | — | Ethoxy- | — | 468 |
| | | | | | | | | ethanol 2- | | |
| 31 | C02:E2 | Form R | Form R | Form A | ML17 | — | — | Cyclo- | — | 421 |
| | | | | | | | | hexanone | | |
| 32 | C02:I2 | Form S + Am | Form B1 + | Form A_plus | ML21 | 50 | 50 | methanol | Xylene-p | 88 |
| | | | Form F1 | | | | | | | |

*the assigned crystalline form is present as well as an amorphous phase; the amorphous could be from a solid amorphous material as well as from a slightly wet material.
**wet solid material.
***a minimum amount of crystalline form is present in mixture with a predominant crystalline form.
^{a}XRPD form assigned after Recrystallization (as wet or dry solid) thus before the stability studies;
^{b}XRPD form assigned after storage of the samples under ambient conditions for 1-1.5 weeks.
^{c}XRPD form assigned after the stability studies (48-72 H, at 40° C./75% RH). [symbol]
indicates data missing or illegible when filed

### Example 4

### Solvent-Drop Grinding Experiments

Table 8 illustrates solvent-drop grinding experiments conducted on the indicated Form. AR42 was subjected to grinding for 30 minutes with a frequency of 30 Hz and with one single drop of the following solvents: Acetonitrile, Isopropanol, 1,2-Dichloroethane, Fluorbenzene, 1,2-Dimethoxyethane, Nitromethane, Propyl acetate, Butyl Acetate, Cyclohexanone, 1,2-Propanediol, Benzonitrile, 1-Octanol. Subsequently, the solids were collected and analysed by XRPD.

TABLE 8

| | Well after | | | | Exp. | |
|---|---|---|---|---|---|---|
| Index | grinding | XRPD Form^{a} | XRPD Form^{b} | XRPD Form^{c} | ID | Screen Solvent |
| 1 | GR2:H1 | Form A | Form A5a | Form A | ML8 | Propyl acetate |
| 2 | GR2:G1 | Form A_min + | Form A5b + | Form A5a | ML7 | Nitromethane |
| | | Form T | Am* | | | |
| 3 | GR2:K1 | Form A_wet** | Form A_wet** | Form A | ML11 | Propanediol 1,2- |
| 4 | GR2:A2 | Form A | Form A | Form A | ML13 | Octanol-1 |
| 5 | GR2:A1 | Form A + | Form A_wet** | Form A | ML1 | — (dry grinding) |
| | | Form B1 | | | | |
| 6 | GR2:E1 | Form A + | Form A | Form A | ML5 | Fluorbenzene |
| | | Form F1 | | | | |
| 7 | GR2:D1 | Form A + | Form A | Form A | ML4 | Dichloroethane 1,2- |
| | | Form F1 | | | | |
| 8 | GR2:J1 | Form A + | Form A | Form A | ML10 | Cyclohexanone |
| | | Form V | | | | |
| 9 | GR2:I1 | Form A3 | Form A | Form A | ML9 | Butyl Acetate |
| 10 | GR2:F1 | Form A5a | Form A5a | Form A5a | ML6 | Dimethoxy ethane, 1,2- |
| 11 | GR2:L1 | Form D1 | Form D1 | Form A | ML12 | Benzonitrile |
| 12 | GR2:C1 | Form H2 | Form A2 | Form A | ML3 | Propanol 2- |
| 13 | GR2:B1 | Form J1 | Form J1 | Form J1 | ML2 | Acetonitrile |
| 14 | GR1:A1 | Form J1 | Form J1 | Am | ML14 | Acetonitrile |

*the assigned crystalline form is present as well as an amorphous phase; the amorphous could be from a solid amorphous material as well as from a slightly wet material.
**wet solid material.
***a minimum amount of crystalline form is present in mixture with a predominant crystalline form.
^{a}XRPD form assigned after Recrystallization (as wet or dry solid) thus before the stability studies;
^{b}XRPD form assigned after storage of the samples under ambient conditions for 1.5-2 weeks.
^{c}XRPD form assigned after the stability studies (46-72 H, at 40° C./75% RH).

### Example 5

### Slurry Conversion Experiments

Tables 9-10 illustrate slurry conversion experiments conducted on the indicated Form. Suspensions of AR42 were prepared by adding and excess of AR42 to the following solvents and solvent mixtures: tert-Butyl methyl ether, Chloroform, Methanol, Tetrahydrofuran, Diisopropyl ether, 2-Methyltetrahydrofuran, Ethanol, Cyclohexane, Acetonitrile, 1,2-Dichloroethane, Fluorbenzene, 1,2-Dimethoxyethane, Diethoxymethane, Hexafluorobenzene, 2,2,4-Trimethylpentane, Water, Methylcyclohexane, Nitromethane, 1,4-Dioxane, 2,2-Dimethyl-3-butanone, 2-Methyl-1-Propanol, Toluene, Octane, Diethyl carbonate, Butyl Acetate, Chlorobenzene, Ethylbenzene, Isopentyl Acetate, Pentyl acetate, Cumene, Anisole, Cyclohexanone, Diethylene glycol dimethyl ether, 1,2,4-Trimethylbenzene, 4-Hydroxy-4-methyl-2-pentanon, 2,6-Dimethyl-4-heptanone. The slurries were stirred at ambient temperature for one week. Subsequently, the samples were centrifuged for 10 minutes at 3000 rpm to separate the liquid from the solid. The solid phase was analysed by XRPD. The solids and liquids were dried under vacuum and analysed by XRPD.

TABLE 9

| | Well after | | | | Exp. | | Conc. |
|---|---|---|---|---|---|---|---|
| Index | slurry | XRPD Form^{a} | XRPD Form^{b} | XRPD Form^{c} | ID | Solvent | (mg/mL) |
| 1 | SL01:G3 | Form A | Form A | Form A | ML32 | Cyclohexanone | 207 |
| 2 | SL01:H1 | Form A | Form A | Form A | ML8 | Cyclohexane | 41 |
| 3 | SL01:C2 | Form A | Form A | Form A | ML15 | Trimethylpentane | 42 |
| | | | | | | 2,2,4- | |
| 4 | SL01:E2 | Form A | Form A + Am* | Form A | ML17 | Methylcyclohexane | 44 |
| 5 | SL01:E1 | Form A | Form A | Form A | ML5 | Diisopropyl ether | 42 |
| 6 | SL01:K2 | Form A | Form A_wet** | Form A_wet** | ML23 | Octane | 41 |
| 7 | SL01:A2 | Form A | Form A_wet** | Form A + Am* | ML13 | Diethoxymethane | 44 |
| 8 | SL01:B2 | Form A | Form A | Form A | ML14 | Hexafluorobenzene | 43 |
| 9 | SL01:J2 | Form A | Form A_wet** | Form A_wet* | ML22 | Toluene | 41 |
| 10 | SL01:F3 | Form A_wet** | Form A_wet** | Form A + Am* | ML31 | Anisole | 41 |
| 11 | SL01:H2 | Form A_wet** | Form A + Am* | Form A + Am* | ML20 | Dimethyl-3-butanone | 43 |
| | | | | | | 2,2- | |
| 12 | SL01:I2 | Form A_wet** | Form A5a + | Form A5a | ML21 | Methyl-1-Propanol 2- | 206 |
| | | | Am_min* | | | | |
| 13 | SL01:I3 | Form A_wet** | Form A6_wet** | Form A5a | ML34 | Trimethylbenzene | 41 |
| | | | | | | 1,2,4- | |
| 14 | SL01:E3 | Form A_wet** | Form A_wet** | Form A + Am* | ML30 | Cumene | 42 |
| 15 | SL01:L2 | Form A_wet** | Form A_wet** | Form A_wet** | ML24 | Diethyl carbonate, | 43 |
| | | | | | | anhydrous | |
| 16 | SL01:K3 | Form A_wet** | Form A + Am* | Form A + Am* | ML36 | Dimethyl-4- | 43 |
| | | | | | | heptanone, 2,6- | |
| 17 | SL01:A1 | Form A_wet** | Form A_low | n | ML1 | tert-Butylmethyl | 43 |
| | | | yield | | | ether, anhydrous | |
| 18 | SL01:A3 | Form A_wet** | Form A_wet** | Form A + Am* | ML25 | Butyl Acetate | 41 |
| 19 | SL01:D3 | Form A_wet** | Form A_wet** | Form A + Am* | ML28 | Isopentyl Acetate | 42 |
| 20 | SL01:J1 | Form A3 + Am* | Form A + | Form A | ML10 | Dichloroethane 1,2- | 44 |
| | | | Form B1 | | | | |
| 21 | SL01:B1 | Form A7 + Am* | Form A3 + Am* | Form A + Am* | ML2 | Chloroform | 41 |
| 22 | SL01:J3 | Form B1_wet** | Form B1 | Form A5a | ML35 | Hydroxy-4-methyl- | 251 |
| | | | | | | 2-pentanon 4- | |
| 23 | SL01:D1 | Form E | Form E | Form A_wet | ML4 | Tetrahydrofuran | 452 |
| 24 | SL01:F1 | Form E | Form A3 | Form A | ML6 | Methyltetrahydro- | 203 |
| | | | | | | furan, 2- | |
| 25 | SL01:K1 | Form F1 | Form A_wet** | Form A | ML11 | Fluorbenzene | 42 |
| 26 | SL01:G2 | Form F4_wet** | Form A + Am* | Form A + Am* | ML19 | Dioxane, 1,4- | 74 |
| 27 | SL01:G1 | Form H1 | Form A | Form A | ML7 | Ethanol absolute | 195 |
| 28 | SL01:I1 | Form J1 | Form J1 | Form J1 | ML9 | Acetonitrite | 44 |
| 29 | SL01:F2 | Form Y + Am* | Form M2 + Am* | Form M2 | ML18 | Nitromethane | 44 |
| 30 | SL01:B3 | Wet sample | Form F3_wet** | Form A + Am* | ML26 | Chlorobenzene | 43 |
| 31 | SL01:C3 | Wet sample | Form F1_wet** | Form A + Am* | ML27 | Ethylbenzene | 42 |
| 32 | SL01:D2 | Wet sample | Form M2_wet** | Form M2 | ML16 | Water | 42 |

*the assigned crystalline form is present as well as an amorphous phase; the amorphous could be from a solid amorphous material as well as from a slightly wet material.
**wet solid material.
***a minimum amount of crystalline form is present in mixture with a predominant crystalline form.
^{a}XRPD from assigned after crystallization experiment (as wet or dry solid) thus before the stability studies;
^{b}XRPD form assigned after storage of the samples under ambient conditions to 1-1.5 weeks.
^{c}XRPD form assigned after the stability studies (48-72 H, at 40° C./75% RH).

TABLE 10

| | Well after | | | | Experiment | | Stock Conc. |
|---|---|---|---|---|---|---|---|
| Index | slurry | XRPD Form^{a} | XRPD Form^{b} | XRPD Form^{c} | ID | Solvent | (mg/mL) |
| 1 | SL02:F2 | Form A | Form A | Form A | ML17 | Methylcyclohexane | 44 |
| 2 | SL02:F3 | Form A | Form A | Form A | ML28 | Isopentyl Acetate | 42 |
| 3 | SL02:F1 | Form A | Form A | Form A | ML6 | Methyltetrahydrofuran, | 203 |
| | | | | | | 2- | |
| 4 | SL02:D2 | Form A | Form A_low | Form A_low | ML15 | Trimethylpentane | 42 |
| | | | yield | yield | | 2,2,4- | |
| 5 | SL02:E1 | Form A | Form A | Form A | ML5 | Diisopropyl ether | 42 |
| 6 | SL02:G3 | Form A | Form A | Form A | ML30 | Cumene | 42 |
| 7 | SL02:J1 | Form A | Form A | Form A | ML10 | Dichloroethane 1,2- | 44 |
| 8 | SL02:K2 | Form A | Form A | Form A | ML22 | Toluene | 41 |
| 9 | SL02:I3 | Form A | Form A | Form A | ML32 | Cyclohexanone | 207 |
| 10 | SL02:H1 | Form A | Form A | Form A | ML8 | Cyclohexane | 41 |
| 11 | SL02:H3 | Form A | Form A | Form A | ML31 | Anisole | 41 |
| 12 | SL02:B2 | Form A | Form A | Form A | ML13 | Diethoxymethane | 44 |
| 13 | SL02:C2 | Form A | Form A | Form A | ML14 | Hexafluorobenzene | 43 |
| 14 | SL02:B3 | Form A | Form A | Form A | ML24 | Diethyl carbonate, | 43 |
| | | | | | | anhydrous | |
| 15 | SL02:A3 | Form A | Form A | Form A | ML23 | Octane | 41 |
| 16 | SL02:A1 | Form A | Form A | Form A | ML1 | tert-Butylmethyl | 43 |
| | | | | | | ether, anhydrous | |
| 17 | SL02:A2 | Form A | Form A | Form A | ML12 | Dimethoxyethane, | 242 |
| | | | | | | 1,2- | |
| 18 | SL02:C3 | Form A + | Form A + | Form A | ML25 | Butyl Acetate | 41 |
| | | Form A3 | Form A3 | | | | |
| 19 | SL02:G1 | Form A + | Form A + | Form A | ML7 | Ethanol absolute | 195 |
| | | Form H3 | Form H3 | | | | |
| 20 | SL02:H2 | Form A3 | Form A4 | Form A | ML19 | Dioxane, 1,4- | 74 |
| 21 | SL02:B1 | Form A4 | Form A4 | Form A5a | ML2 | Chloroform | 41 |
| 22 | SL02:J2 | Form A5a | Form A5a | Form A5a | ML21 | Methyl-1-Propanol 2- | 206 |
| 23 | SL02:I2 | Form A6 | Form A6 | —^{d} | ML20 | Dimethyl-3-butanone | 43 |
| | | | | | | 2,2- | |
| 24 | SL02:L3 | Form B1 | Form B1 | Form A | ML35 | Hydroxy-4-methyl-2- | 251 |
| | | | | | | pentanon 4- | |
| 25 | SL02:D1 | Form E | Form E | Form A | ML4 | Tetrahydrofuran | 452 |
| 26 | SL02:L1 | Form F1 | Form A + | Form A | ML11 | Fluorbenzene | 42 |
| | | | Form F1 | | | | |
| 27 | SL02:D3 | Form F3 | Form F3 | Form A | ML26 | Chlorobenzene | 43 |
| 28 | SL02:I1 | Form J1 | Form J1 | Form J1 | ML9 | Acetonitrile | 44 |
| 29 | SL02:C1 | Form M1 | Form M1 | Form M1 | ML3 | methanol | 510 |
| 30 | SL02:E2 | Form M2 | Form M2 | Form M2 | ML16 | Water | 42 |
| 31 | SL02:G2 | Form M2 + Am | Form M2 + Am | Form M2 | ML18 | Nitromethane | 44 |
| 32 | SL02:E3 | Form S | Form A + | Form A | ML27 | Ethylbenzene | 42 |
| | | | Form F1 | | | | |
| 33 | SL02:A4 | Form U1 + Am | Form U1 + Am | Form A + Am | ML36 | Dimethyl-4- | 43 |
| | | | | | | heptanone, 2,6- | |
| 34 | SL02:K3 | Form U1 + Am | n | Form A + Am | ML34 | Trimethylbenzene | 41 |
| | | | | | | 1,2,4- | |
| 35 | SL03:A5 | — | Form A6 | Form A5b | [symbol] | Dimethyl-3-butanone | [symbol] |
| | | | | | ML20 | 2,2- | 43 |
| 36 | SL07:A8/ | Form A_wet | Form A | —^{e} | ML29 | Pentyl acetate | 139 |
| | SL03:C8 | | | | | | |

*the assigned crystalline form is present as well as an amorphous phase; the amorphous could be from a solid amorphous material as well as from a slightly wet material.
**wet solid material.
***a minimum amount of crystalline form is present in mixture with a predominant crystalline form.
^{a}XRPD form assigned after crystallization experiment (as wet or dry solid) thus before the stability studies;
^{b}XRPD form assigned after storage of the samples under ambient conditions for 1-1.5 weeks.
^{c}XRPD form assigned after the stability studies (48-72 H, at 40° C./75% RH).
^{d}see entry#35 in Table 10 for the XRPD form after stability studies.
^{e}— no stability studies were carried out. [symbol]
indicates data missing or illegible when filed

Although the above description refers to particular aspects, it is to be understood that these aspects are merely illustrative. It will be apparent to those skilled in the art that various modifications and variations can be made to the polymorphic forms and methods described herein. Thus, it is intended that the present description include modifications and variations that are within the scope of the appended claims and their equivalents.

What is claimed is:

1. An AR-42 crystalline form characterized by the X-ray powder diffraction pattern of FIG. 3A.

2. An AR-42 crystalline form characterized by the X-ray powder diffraction pattern of FIG. 3B.

3. An AR-42 crystalline form characterized by the X-ray powder diffraction pattern of FIG. 3C.

4. An AR-42 crystalline form characterized by the X-ray powder diffraction pattern of FIG. 3D.

5. An AR-42 crystalline form characterized by the X-ray powder diffraction pattern of FIG. 3E.

6. An AR-42 crystalline form characterized by the X-ray powder diffraction pattern of FIG. 3F.

7. An AR-42 crystalline form characterized by the X-ray powder diffraction pattern of FIG. 3G.

8. An AR-42 crystalline form characterized by the X-ray powder diffraction pattern of FIG. 3H.

9. An AR-42 crystalline form characterized by the X-ray powder diffraction pattern of FIG. 3I.

10. An AR-42 crystalline form characterized by the X-ray powder diffraction pattern of FIG. 3J.

11. An AR-42 crystalline form characterized by the X-ray powder diffraction pattern of FIG. 3K.

12. An AR-42 crystalline form characterized by the X-ray powder diffraction pattern of FIG. 5A.

13. An AR-42 crystalline form characterized by the X-ray powder diffraction pattern of FIG. 5B.

14. An AR-42 crystalline form characterized by the X-ray powder diffraction pattern of FIG. 5C.
An AR-42 crystalline form characterized by the X-ray powder diffraction pattern of FIG. 5D.

15. An AR-42 crystalline form characterized by the X-ray powder diffraction pattern of FIG. 5E.

16. An AR-42 crystalline form characterized by the X-ray powder diffraction pattern of FIG. 5F.

17. An AR-42 crystalline form characterized by the X-ray powder diffraction pattern of FIG. 7A.

18. An AR-42 crystalline form characterized by the X-ray powder diffraction pattern of FIG. 7B.

19. An AR-42 crystalline form characterized by the X-ray powder diffraction pattern of FIG. 7C.
An AR-42 crystalline form characterized by the X-ray powder diffraction pattern of FIG. 7D.

20. An AR-42 crystalline form characterized by the X-ray powder diffraction pattern of FIG. 7E.

21. An AR-42 crystalline form characterized by the X-ray powder diffraction pattern of FIG. 7F.

22. An AR-42 crystalline form characterized by the X-ray powder diffraction pattern of FIG. 7G.

23. An AR-42 crystalline form characterized by the X-ray powder diffraction pattern of FIG. 7H.

24. An AR-42 crystalline form characterized by the X-ray powder diffraction pattern of FIG. 9A.

25. An AR-42 crystalline form characterized by the X-ray powder diffraction pattern of FIG. 9B.

26. An AR-42 crystalline form characterized by the X-ray powder diffraction pattern of FIG. 9C.

27. An AR-42 crystalline form characterized by the X-ray powder diffraction pattern of FIG. 9D.

28. An AR-42 crystalline form characterized by the X-ray powder diffraction pattern of FIG. 9E.

29. An AR-42 crystalline form characterized by the X-ray powder diffraction pattern of FIG. 9F.

30. An AR-42 crystalline form characterized by the X-ray powder diffraction pattern of FIG. 9G.

31. An AR-42 crystalline form characterized by the X-ray powder diffraction pattern of FIG. 11A.

32. An AR-42 crystalline form characterized by the X-ray powder diffraction pattern of FIG. 11B.

33. An AR-42 crystalline form characterized by the X-ray powder diffraction pattern of FIG. 11C.

34. An AR-42 crystalline form characterized by the X-ray powder diffraction pattern of FIG. 11D.

35. An AR-42 crystalline form characterized by the X-ray powder diffraction pattern of FIG. 11E.

36. A pharmaceutical composition comprising the AR-42 crystalline form of any one of claims 1-35 and a pharmaceutically acceptable excipient, diluent, or carrier.

37. The pharmaceutical composition of claim 36, in a unit dosage form.

38. The pharmaceutical composition of claim 37, wherein the unit dosage form is selected from the group consisting of tablets, pills, capsules, and troches.

39. The pharmaceutical composition of claim 36, where the AR-42 crystalline form is present in an amount from about 5 to about 100 mg.

40. The pharmaceutical composition of claim 36, further comprising at least one additional active pharmaceutical agent.

41. The pharmaceutical composition of claim 40, wherein the additional active pharmaceutical agent is selected from the group consisting of an antitumor agent, a hormone, a steroid, or a retinoid.

42. The pharmaceutical composition of claim 41, wherein the additional active ingredient is selected from the group consisting of alkylating agent, an antimetabolite, a hormonal agent, an antibiotic, colchicine, a vinca alkaloid, L-asparaginase, procarbazine, hydroxyurea, mitotane, nitrosoureas, and an imidazole carboxamide.

43. A method of treating a mammal, comprising administering a therapeutically effective amount of the pharmaceutical composition of claims 36-42 to a mammal in need of treatment.

44. The method of claim 43, wherein the therapeutically effective amount is from about 5 to about 100 mg.

45. The method of claim 43, wherein the pharmaceutical composition is administered to the mammal by a route of administration selected from the group consisting of orally, parenterally, sublingually, intranasally, intrathecally, topically, or rectally.

46. The method of claim 43, the method of claim 43 where the mammal is a human.

47. A method of making an AR-42 polymorphic form comprising combining AR-42 with a solvent selected from the group consisting of methycyclohexane, isopentyl acetate, n-ethyl tetrahydrofuran-2, trimethylpentane 2,2,4-diisopropyl ether, cumene, dichloroethane 1,2-, toluene, cyclohexanone, cyclohexane, anisole, diethyl carbonate (anyhydrous), octane, tert-butylmethyl ether (anhydrous), dimethoxyethane 1,2-, butyl acetate, absolute ethanol, dioxane, 1,4-, chloroform, methyl-1-propanol 2-, dimethyl-3-butanone 2,2-, hydroxy-4-methyl-2-pentanon 4-, tetrahydrofuran, fluorbenzene, chlorobenzene, acetonitrile, methanol, water, nitromethane, ethylbenzene, dimethyl-4-heptanone, 2,6-, trimethylbenzene 1,2,4-, dimethyl-3-butanone 2,2-, nitromethane, fluorobenzene, dioxane, 1,4, methyltetrahydrofuran, 2-, and pentyl acetate.
